(19) United States
(12) Patent Application Publication
(10) Pub. No.: US 2016/0327551 A1
(43) Pub. Date: Nov. 10, 2016

(54) SERS NANOPARTICLES AND METHODS FOR USING THE SAME

(71) Applicant: BECTON, DICKINSON AND COMPANY, Franklin Lake, NJ (US)

(72) Inventors: Majid Mehrpouyan, Gilroy, CA (US); Ming Yan, Pleasanton, CA (US)

(21) Appl. No.: 15/108,555

(22) PCT Filed: Jan. 23, 2015
(86) PCT No.: PCT/US15/12648

§ 371 Date: Jun. 27, 2016

Related U.S. Application Data
(60) Provisional application No. 61/940,151, filed on Feb. 14, 2014.
(60) Provisional application No. 62/023,703, filed on Jul. 11, 2014.

(51) Int. Cl. G01N 33/543; G01N 33/58; G01N 21/65; C09K 11/06

(52) U.S. Cl. CPC G01N 33/54346; C09K 11/06; G01N 33/587; G01N 21/658; G01N 33/54393; C09K 2211/1088

(57) ABSTRACT

Provided are SERS nanoparticles. In certain aspects, the SERS nanoparticles are fluorescently labeled SERS nanoparticles that include a SERS nanoparticle and a fluorescent label stably associated with a surface of the SERS nanoparticle. Aspects of the present disclosure further include methods of using the SERS nanoparticles, e.g., in a variety of different applications, as well as kits that find use in practicing embodiments of the methods.

### CROSS-REFERENCE TO RELATED APPLICATIONS

This application is related to U.S. Provisional Patent Application Ser. No. 61/940,151, filed Feb. 14, 2014, and U.S. Provisional Patent Application Ser. No. 62/023,703, filed Jul. 11, 2014, the disclosures of which applications are incorporated herein by reference.

### INTRODUCTION

Surface-enhanced Raman spectroscopy (SERS) is a technique that enhances the Raman scattering effect by observing molecules adsorbed onto rough metal surfaces or metallic colloids. SERS is a laser-based optical spectroscopy protocol that generates a fingerprint-like vibrational spectrum having features that are much narrower than typical fluorescence. Monochromatic far-red or near-IR light, which have photon energies that are insufficient to produce the inherent background fluorescence in biological samples, may be used to elicit Raman scattering.

Raman scattering generally refers to the inelastic scattering of a photon incident on a molecule. Photons that are inelastically scattered have an optical frequency (vᵢ), which is different than the frequency of the incident light (vₒ). The difference in energy (ΔE) between the incident light and the inelastically scattered light can be represented as (ΔE)=h|vₒ−vᵢ|, wherein h is Planck's constant, and corresponds to energies that are absorbed by the molecule. The incident radiation can be of any frequency vₒ, but typically is monochromatic radiation in the visible or near-infrared spectral region. The absolute difference |vₒ−vᵢ| is an infrared, e.g., vibrational, frequency. The frequency vᵢ of the “Raman scattered” radiation can be greater than or less than vₒ, but the amount of light with frequency vᵢ<vₒ (Stokes radiation) is greater than that with frequency vᵢ>vₒ (anti-Stokes radiation).

The Raman spectra typically cover vibrational energies from 300-3500 cm⁻¹, making it possible to measure multiple (e.g., a dozen or more) tags simultaneously using a single light source. Normal Raman spectra are weak, however, thus precluding their use for applications such as single molecule detection. In SERS, molecules in sufficiently close proximity to nanoscale roughness features on surfaces (e.g., noble metal surfaces (e.g., gold, silver, copper, platinum or the like)) exhibit million- to trillion-fold increases in scattering efficiency. Such an increase is known as the enhancement factor (EF) and allows SERS signals to be used for the detection of single molecules.

### SUMMARY

Provided are SERS nanoparticles. In certain aspects, the SERS nanoparticles are fluorescently labeled SERS nanoparticles that include a SERS nanoparticle and a fluorescent label stably associated with a surface of the SERS nanoparticle. Aspects of the present disclosure further include methods of using the SERS nanoparticles, e.g., in a variety of different applications, as well as kits that find use in practicing embodiments of the methods.

### BRIEF DESCRIPTION OF THE FIGURES

FIG. 1 schematically illustrates the preparation of SERS nanoparticles according to one embodiment of the present disclosure.

FIG. 2A provides transmission electron microscopy (TEM) images of SERS nanoparticles according to one embodiment of the present disclosure. FIG. 2B shows the dynamic light scattering size distribution of the particles shown in FIG. 2A.

FIG. 3 schematically illustrates the preparation of a fluorescently labeled SERS nanoparticle according to one embodiment of the present disclosure.

FIGS. 4A and 4B provide flow cytometric data. Panel A shows control flow cytometric data for an antibody conjugate, while panel B shows flow cytometric data for a fluorescently labeled SERS nanoparticle according to one embodiment of the present disclosure.

FIG. 5 schematically illustrates the preparation of an antibody-labeled SERS nanoparticle without a blocking agent coating on the surface of the SERS nanoparticle according to one embodiment of the present disclosure.

FIG. 6 provides Raman imaging data from antibody-labeled SERS nanoparticles prepared as shown in FIG. 5.

FIG. 7 provides data showing the relative Raman signal intensities of the images shown in FIG. 6.

FIG. 8 provides Raman imaging data from fluorescently labeled SERS nanoparticles prepared as shown in FIG. 3.

FIG. 9 provides data showing the relative Raman signal intensities of the images shown in FIG. 8.

FIG. 10 provides cell sorting data from whole blood stained with fluorescently labeled SERS nanoparticles according to one embodiment of the present disclosure.

FIG. 11 provides Raman imaging data of ten independent cells sorted after staining with the fluorescently labeled SERS nanoparticles according to the embodiment shown in FIG. 10.

### DETAILED DESCRIPTION

Provided are SERS nanoparticles. In certain aspects, the SERS nanoparticles are fluorescently labeled SERS nanoparticles that include a SERS nanoparticle and a fluorescent label stably associated with a surface (e.g., an outer silica shell surface) of the SERS nanoparticle. Aspects of the present disclosure further include methods of using the SERS nanoparticles, e.g., in a variety of different applications, as well as kits that find use in practicing embodiments of the methods.

Before the nanoparticles and methods of the present disclosure are described in greater detail, it is to be understood that the nanoparticles and methods are not limited to particular embodiments described, as such may, of course, vary. It is also to be understood that the terminology used herein is for the purpose of describing particular embodiments only, and is not intended to be limiting, since the scope of the nanoparticles and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the nanoparticles and methods. The upper and lower limits of these smaller ranges may independently be included in the smaller ranges and are also encompassed within the nanoparticles and methods, subject to any specifically excluded limit in the stated range. Where the stated range includes one or both of the limits, ranges excluding either or both of those included limits are also included in the nanoparticles and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the nanoparticles and methods belong. Although any nanoparticles and methods similar or equivalent to those described herein can also be used in the practice or testing of the nanoparticles and methods, representative illustrative nanoparticles, methods and materials are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present nanoparticles and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the nanoparticles and methods, which are, for clarity, described in the context of separate embodiments, may also be provided in combination in a single embodiment. Conversely, various features of the nanoparticles and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions/kits. In addition, all sub-combinations listed in the embodiments describing such variables are also specifically embraced by the present nanoparticles and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Nanoparticles

The present disclosure provides SERS nanoparticles. In certain aspects, the SERS nanoparticles are fluorescently labeled SERS nanoparticles that include a SERS nanoparticle and a fluorescent label stably associated with a surface (e.g., an outer silica shell surface) of the SERS nanoparticle. Aspects of the SERS nanoparticles will now be described in detail.

As summarized above, fluorescently labeled SERS nanoparticles of the present disclosure include a SERS nanoparticle. By “nanoparticle” is meant a particle having at least one dimension in the range of from 1 nm to 1000 nm, from 20 nm to 750 nm, from 50 nm to 500 nm, including 100 nm to 300 nm, e.g., 120-200 nm. The SERS nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain aspects, the SERS nanoparticle is a spherical or spheroid particle having a diameter of from 50 to 500 nm, e.g., from 100 to 300 nm.

As used herein, a “SERS nanoparticle” is a nanoparticle having a surface that induces, causes, or otherwise supports surface-enhanced Raman light scattering (SERS) or surface-enhanced resonance Raman light scattering (SERS). A number of surfaces are capable of producing a SERS signal, including roughened surfaces, textured surfaces, and other surfaces, including smooth surfaces. In certain aspects, the surface of the SERS nanoparticle that induces, causes, or otherwise supports SERS is an internal surface of the SERS nanoparticle (e.g., a surface of a core of the SERS nanoparticle). The core may be made of any suitable material for providing a surface that supports SERS. In certain aspects, the core is made of a metal. Suitable metals include, but are not limited to, Group 11 metals, such as Cu, Ag, and Au, or any other metals that support SERS, such as alkali metals. According to certain embodiments, the core includes a single metal element (e.g., Cu, Ag, Au, or the like). In other embodiments, the core includes a combination of at least two elements, such as an alloy, for example, a binary alloy. The core of the SERS nanoparticle may be solid, semi-porous, porous, or hollow. In certain aspects, the core is magnetic.

The surface of the SERS nanoparticle that supports SERS has adsorbed thereon or in close proximity to (e.g., within about 50 angstroms) a SERS reporter molecule. By “SERS reporter molecule” is meant any molecule or chemical compound that is capable of producing a Raman spectrum when it is illuminated with radiation of an appropriate wavelength. A “SERS reporter molecule” also can be referred to herein as a “Raman-active molecule” or “SERS-active molecule,” which can be used interchangeably.

The SERS reporter molecule may be any molecule capable of producing a Raman spectrum. According to certain embodiments, the SERS reporter molecule is selected from 4-mercaptopyridine (4-MP), trans-4,4′bis(pyridyl)ethylene (BPE), quinolinethiol, 1,4-phenyldiisocyanide, mercaptobenzamidazole, 4-cyanopyridine, 1′,3,3,3′,3′-hexamethylindotricarbocyanine iodide, 3,3′-diethyltiatricarbocyanine, malachite green isothiocyanate, bis-(pyridyl)acetylenes, Bodipy, 4,4′-dipyridyl (DIPY), D8-4,4′-dipyridyl (d8DIPY), trans-1,2-bis(4-pyridyl)-ethylene (BPE), quinoline thiol, 2-quinolinethiol (QSH), 1,2-dil(4-pyridyl)acetylene (BPA), 4-azobis(pyridine) (4-AZP), GM19, 1-(4-pyridyl)-1-cyano-2-(2-fluoro-4-pyridyl)-ethylene (CNFBPE), 1-cyano-1-(4-quinolinyl)-2-(4-pyridyl)-ethylene (CQPE), dye 10, 4-(4-hydroxyphenylazo)pyridine, and CyNAMLA-381.

In certain aspects, the SERS nanoparticle includes a core (e.g., a metal core, such as a core made of Au, Ag and/or Cu), Raman reporter molecules adsorbed to or in close proximity to the surface of the core, and a “shell” (e.g., a glass shell, such as a silica shell) disposed over the core which encapsulates the Raman reporter molecules. SERS nanoparticles having a core, Raman reporters, and a shell encapsulating the Raman reporters are available and include SERS nanotags from Oxonica Materials, Inc. (Mountain View, Calif.). See, e.g., Zavaleta et al. (2009) PNAS 106(32):13511-13516, US Application Publication No. US2011/0172523, US Application Publication No. US2012/0164624, and International Application Publication No. WO2013/165615, the disclosures of which applications are incorporated herein by reference in their entireties for all purposes.

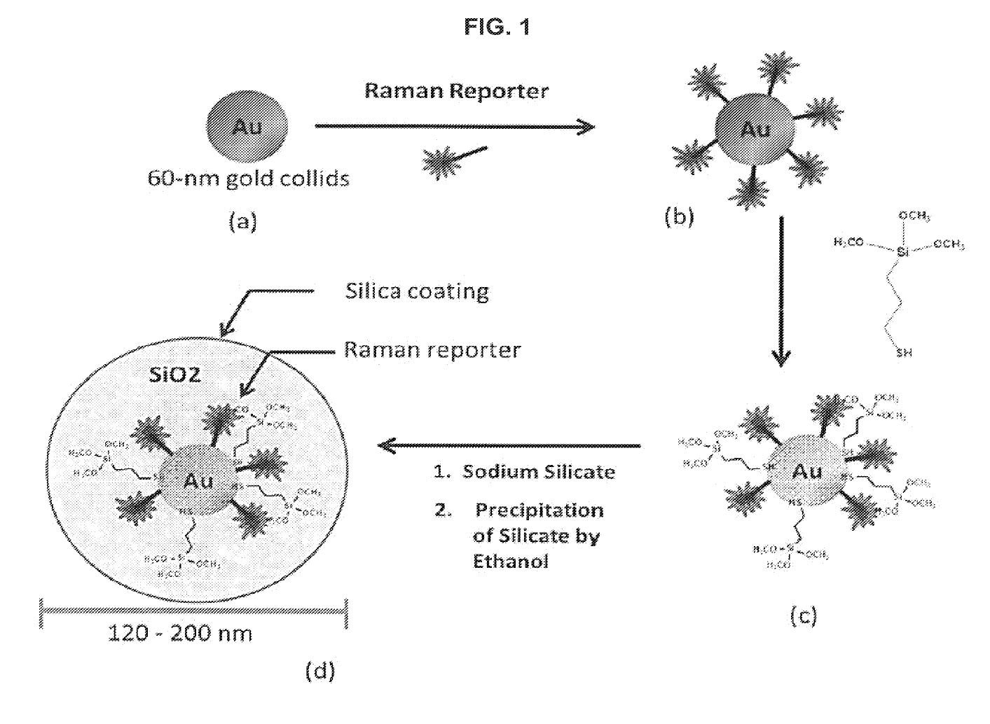

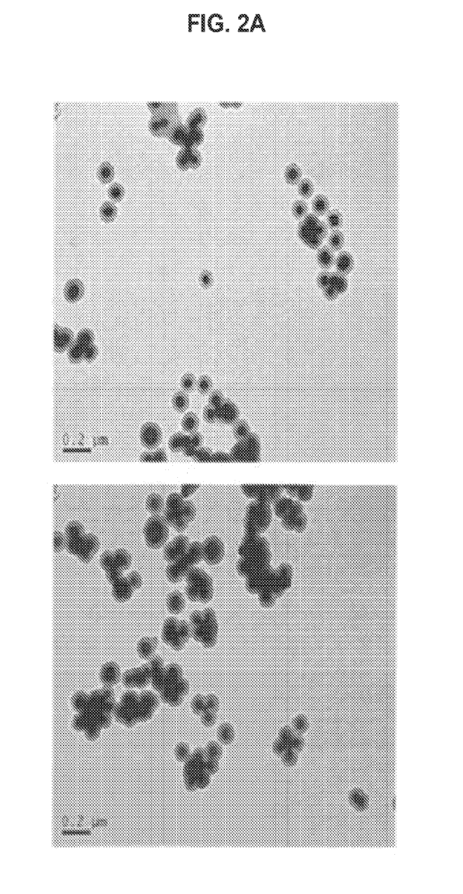

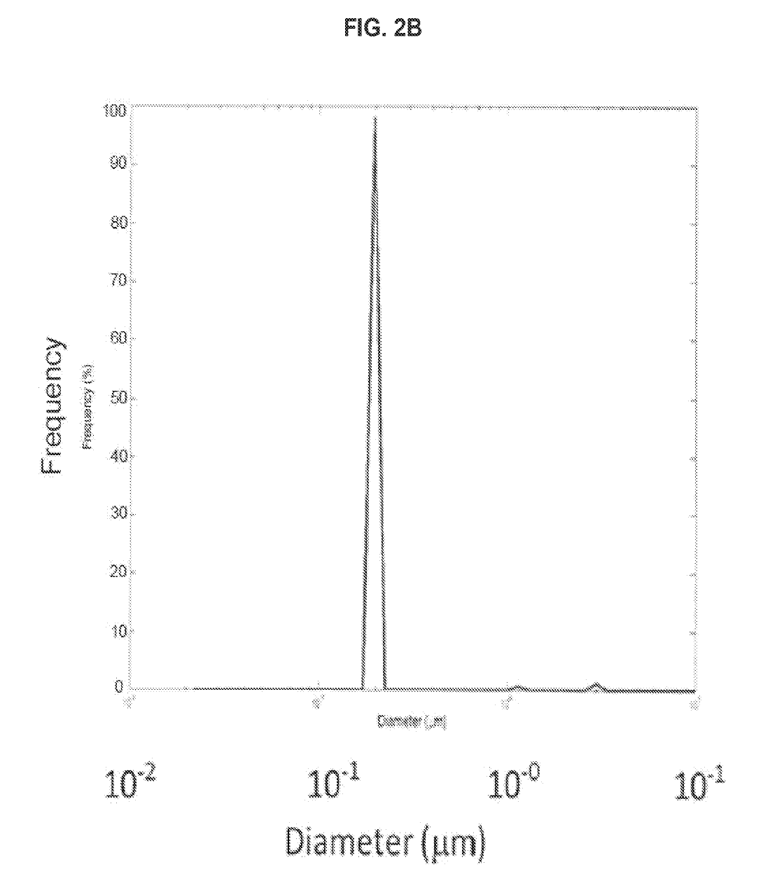

A strategy for producing a SERS nanoparticle for use in the fluorescently labeled SERS nanoparticles of the present disclosure is schematically illustrated in FIG. 1. The first step according to this example scheme is the adsorption of Raman reporter molecules to the surface of a core nanoparticle (in this example, a spheroid gold nanoparticle core having a diameter of 50-60 nm). The resulting core nanoparticle is then treated with (3-mercaptopropyl)trimethoxysilane and sodium silicate, followed by precipitation of silicate by ethanol, yielding a SERS nanoparticle having a core-shell structure with a diameter of about 120-200 nm. When prepared in this way, the (3-mercaptopropyl)trimethoxysilane provides thiol functional groups on the external surface of the SERS nanoparticle, which may facilitate attachment of a blocking agent and/or specific binding member to the external surface of the SERS nanoparticle as will be described in more detail below. FIG. 2 shows transmission electron microscopy (TEM) images (Panel A) and hydrodynamic size distribution (Panel B) of SERS nanoparticles produced according to the scheme shown in FIG. 1. The core-shell structure is clearly visible in the TEM images.

As summarized above, the fluorescently labeled SERS nanoparticles of the present disclosure include a fluorescent label stably associated with a surface (e.g., the external-most surface, such as the outer surface of a silica shell) of the SERS nanoparticle. By “stably associated” is meant a physical association between two entities in which the mean half-life of association is one day or more in PBS at 4° C. In certain aspects, the physical association between the two entities has a mean half-life of one day or more, one week or more, one month or more, including six months or more, e.g., 1 year or more, in PBS at 4° C. According to certain embodiments, the stable association arises from a covalent bond between the two entities, a non-covalent bond between the two entities (e.g., an ionic or metallic bond), or other forms of chemical attraction, such as hydrogen bonding, Van der Waals forces, and the like.

In certain aspects, the fluorescently labeled SERS nanoparticle includes a fluorescent label bound directly to a surface of the SERS nanoparticle. By “directly” is meant any type of stable association (e.g., a covalent linkage, a non-covalent linkage, etc.), but excluding an intervening element such as a blocking agent, a specific binding member, or the like. According to certain embodiments, the surface to which the fluorescent label is directly attached is the external-most surface of the SERS nanoparticle, e.g., the fluorescent label is directly attached to the outer surface of the shell of a SERS nanoparticle having a core-shell structure.

According to certain embodiments, the fluorescently labeled SERS nanoparticle includes a fluorescent label bound indirectly thereto. As used herein, “indirectly” means not “directly” as defined above. For example, a fluorescent label is indirectly bound to a surface of the SERS nanoparticle when the fluorescent label is bound to a blocking agent or a specific binding member, and the blocking agent or specific binding member is bound directly or indirectly to a surface of the SERS nanoparticle via an entity other than the fluorescent label. In certain aspects, the surface to which the fluorescent label is indirectly attached is the external-most surface of the SERS nanoparticle, e.g., the fluorescent label is indirectly attached to the outer surface of the shell of a SERS nanoparticle having a core-shell structure.

In certain aspects, the fluorescent label is indirectly bound to a surface of the SERS nanoparticle by stable association of a blocking agent or a specific binding member with the surface, where the blocking agent or specific binding member includes the fluorescent label, e.g., the blocking agent or specific binding member has a fluorescent label bound thereto (e.g., directly or indirectly bound thereto). In certain aspects, both a blocking agent and a specific binding member are stably associated with a surface of the SERS nanoparticle (e.g., the outer surface of the shell of a SERS nanoparticle having a core-shell structure), where each of the blocking agent and the specific binding include a fluorescent label.

When the fluorescently labeled SERS nanoparticle includes a blocking agent and/or specific binding member stably associated with a surface thereof (e.g., the outer surface of the shell of a SERS nanoparticle having a core-shell structure), any desirable configuration may be employed. For example, a blocking agent that includes a fluorescent label may be directly bound to a surface of the SERS nanoparticle (e.g., the outer surface of the shell of a SERS nanoparticle having a core-shell structure), thereby stably associating the fluorescent label to the surface. Similarly, a specific binding member that includes a fluorescent label may be directly bound to a surface of the SERS nanoparticle (e.g., the outer surface of the shell of a SERS nanoparticle having a core-shell structure), thereby stably associating the fluorescent label to the surface.

According to certain embodiments, a blocking agent that does not include a fluorescent label is directly bound to a surface of the SERS nanoparticle (e.g., the outer surface of the shell of a SERS nanoparticle having a core-shell structure), and a specific binding member that includes a fluorescent label is bound to the blocking agent, thereby stably associating the fluorescent label to the surface. In certain aspects, a specific binding member that does not include a fluorescent label is directly bound to a surface of the SERS nanoparticle (e.g., the outer surface of the shell of a SERS nanoparticle having a core-shell structure), and a blocking agent that includes a fluorescent label is bound to the specific binding member, thereby stably associating the fluorescent label to the surface.

In certain aspects, a blocking agent that includes a fluorescent label is directly bound to a surface of the SERS nanoparticle (e.g., the outer surface of the shell of a SERS nanoparticle having a core-shell structure), and a specific binding member that includes a fluorescent label is bound to the blocking agent, thereby stably associating two or more fluorescent labels to the surface. Alternatively, a specific binding member that includes a fluorescent label may be directly bound to a surface of the SERS nanoparticle (e.g., the outer surface of the shell of a SERS nanoparticle having a core-shell structure), and a blocking agent that includes a fluorescent label is bound to the specific binding member, thereby stably associating two or more fluorescent labels to the surface.

In certain aspects, the fluorescently labeled SERS nanoparticle includes a SERS nanoparticle and a fluorescent label stably associated with a surface of the SERS nanoparticle (e.g., the outer surface of the shell of a SERS nanoparticle having a core-shell structure), where the SERS nanoparticle includes a blocking agent stably associated with a surface of the SERS nanoparticle. Such a SERS nanoparticle may include a specific binding member stably associated with a surface of the SERS nanoparticle (e.g., the outer surface of the shell of a SERS nanoparticle having a core-shell structure), where at least one of the blocking agent and the specific binding member includes a fluorescent label. Both of the blocking agent and the specific binding member may include a fluorescent label.

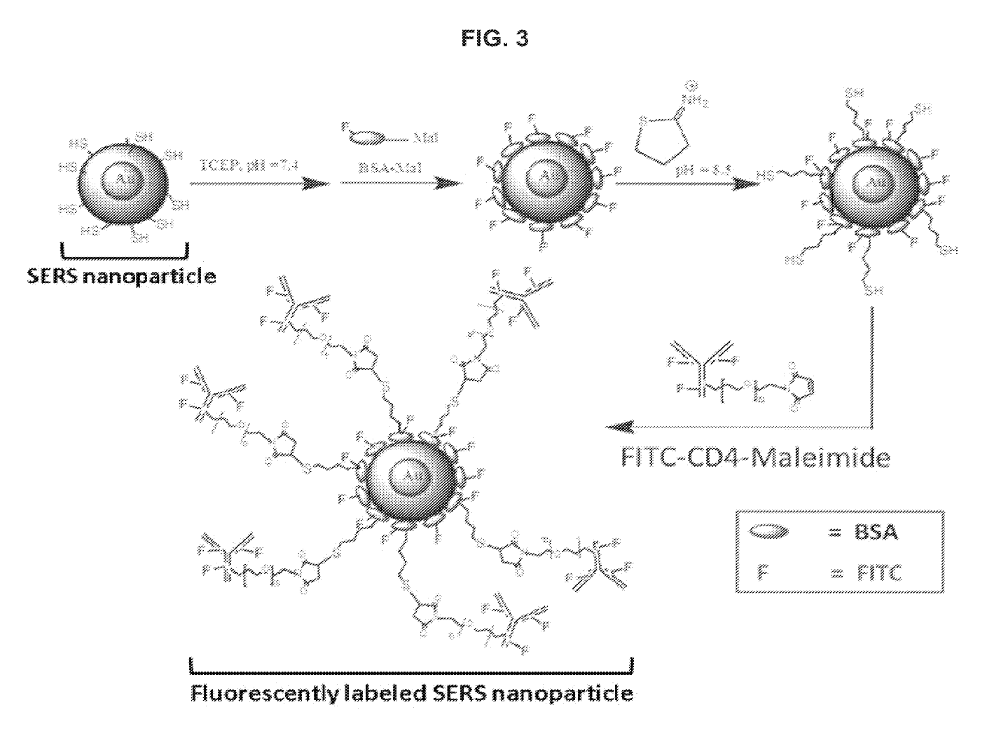

A plurality of blocking agents and/or specific binding members, where the plurality of blocking agents and/or specific binding members are labeled (e.g., fluorescently labeled), may be stably associated with a surface of the SERS nanoparticle (e.g., the outer surface of the shell of a SERS nanoparticle having a core-shell structure). A fluorescently labeled SERS nanoparticle according to one such an embodiment is schematically illustrated in FIG. 3. Shown is an example fluorescently labeled SERS nanoparticle that includes a plurality of fluorescent labeled blocking agents (in this example, FITC labeled bovine serum albumin (BSA)) directly bound to the external surface of a SERS nanoparticle having a core-shell structure (in this example, a gold core and silica shell, e.g., as shown in FIG. 1, Panel (d)). In this way, the SERS nanoparticle includes a coating of fluorescent labeled blocking agents directly bound to its external surface. According to the embodiment shown in FIG. 3, fluorescent labeled specific binding members (in this example, FITC labeled anti-CD4 antibodies) are directly bound (in this example, via PEG linkers) to the blocking agents directly bound to the outer surface of the silica shell of the SERS nanoparticle. The embodiment shown in FIG. 3 is one example of a fluorescently labeled SERS nanoparticle provided by the present disclosure that is highly fluorescent.

Aspects of the present disclosure include labeled SERS nanoparticles having a label stably associated with a surface of a SERS nanoparticle (e.g., the outer surface of the shell of a SERS nanoparticle having a core-shell structure). As used herein, the terms “label” and “detectable label” refer to a molecule capable of detection, including, but not limited to, radioactive isotopes, fluorescent labels, magnetic labels, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like.

The term “fluorescent label” refers to a substance or a portion thereof which is capable of exhibiting fluorescence in the detectable range. A fluorescent label stably associated with the surface of the SERS nanoparticle may be any fluorescent label that finds use in a particular application of interest. Fluorescent labels of interest include, but are not limited to, fluorescein and its derivatives (e.g., fluorescein isothiocyanate (FITC)); rhodamine and its derivatives; cyanine and its derivatives; coumarin and its derivatives; Cascade Blue and its derivatives; Lucifer Yellow and its derivatives; BODIPY and its derivatives; and the like. Fluorescent labels of interest also phycoerythrin (PE), R-phycoerythrin (R-PE), indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like.

As summarized above, aspects of the present disclosure include fluorescently labeled SERS nanoparticles having a blocking agent stably associated with the surface of the SERS nanoparticle. By “blocking agent” is meant any biochemical or non-biochemical agent that finds use in reducing non-specific binding to (or by) the fluorescently labeled SERS nanoparticle in an application of interest, e.g., a research or clinical (e.g., clinical diagnostic) assay of interest. In certain aspects, the blocking agent is a protein, a nucleic acid, a carbohydrate, a natural molecule or polymer, a synthetic molecule or polymer, a fluorescent molecule or polymer, or any combination thereof. According to certain embodiments, the blocking agent is a protein blocking agent. Protein blocking agents of interest include, but are not limited to, protein blocking agents derived from a blood component (e.g., a serum protein or a plasma protein) or recombinant versions thereof. Such blocking agents include, but are not limited to, albumin, recombinant albumin, Albumin-DX LR, bovine serum albumin (BSA), polymerized bovine serum albumin (pBSA), human serum albumin (HSA), polymerized human serum albumin (pHSA), a purified and/or recombinant immunoglobulin (e.g., a purified and/or recombinant animal immunoglobulin), and the like. Other useful blocking agents include proteins present in milk (e.g., mammalian milk), such as a milk phosphoprotein. According to certain embodiments, the milk phosphoprotein is casein. Any other suitable blocking agent may be employed, such as lysine, polyethylene glycol (PEG), etc. for stable association to the surface of a SERS nanoparticle according to the present disclosure.

As summarized above, aspects of the present disclosure include fluorescently labeled SERS nanoparticles having a specific binding member stably associated with the surface of the SERS nanoparticle. The term “binding member” as used herein refers to any agent (e.g., a protein (e.g., antibody), small molecule, and the like) that specifically binds to a target analyte. The terms “specific binding,” “specifically binds,” and the like, refer to the preferential binding to a molecule relative to other molecules or moieties in a solution or reaction mixture. In some embodiments, the affinity between binding member and the target analyte to which it specifically binds when they are specifically bound to each other in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower K_{d}. As such, “binds specifically” or “specifically binds” is not meant to preclude a given binding member from binding to more than one analyte of interest. For example, antibodies that bind specifically to an analyte polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g., by use of appropriate controls.

The specific binding member may be any agent that specifically binds to an analyte of interest. For example, the specific binding member may be a receptor (e.g., when the analyte of interest is a ligand of the receptor); a ligand (e.g., when the analyte of interest is receptor for the ligand); a nucleic acid (e.g., when the analyte of interest is a nucleic acid complementary thereto); a substrate (e.g., when the analyte of interest is an enzyme that binds the substrate); an enzyme (e.g., when the analyte of interest is a substrate to which the enzyme binds); streptavidin (e.g., when the analyte of interest includes a biotin moiety); biotin (e.g., when the analyte of interest includes a streptavidin moiety); and any other specific binding member useful for binding a target analyte in an application of interest.

In certain aspects, the specific binding member is an antibody, or antigen-binding fragment thereof. As used herein, the term “antibodies” includes antibodies or immunoglobulins of any isotype, fragments of antibodies which retain specific binding to antigen, including, but not limited to, Fab, Fv, scFv, and Fd fragments, chimeric antibodies, humanized antibodies, fully human antibodies, single-chain antibodies, and fusion proteins comprising an antigen-binding portion of an antibody and a non-antibody protein. The antibodies may be further conjugated to other moieties, such as members of specific binding pairs, e.g., biotin (member of biotin-avidin specific binding pair), and the like. Also encompassed by the term are Fab′, Fv, F(ab′)₂, and or other antibody fragments that retain specific binding to antigen, and monoclonal antibodies. In other aspects, the binding members may be antigens, where the analytes of interest are antibodies.

Also provided by the present disclosure are methods of making the fluorescently labeled SERS nanoparticles of the present disclosure. The methods include stably associating a fluorescent label with a surface of a SERS nanoparticle (e.g., the outer surface of the shell (e.g., silica shell) of a SERS nanoparticle having a core-shell structure). The fluorescent label may be stably associated according to any of the configurations described elsewhere herein, e.g., directly to the outer surface of the SERS nanoparticle, indirectly via a blocking agent and/or a specific binding member, or the like. According to one embodiment, stably associating a fluorescent label with a surface of a SERS nanoparticle includes stably associating a blocking agent and a specific binding member with the surface of the SERS nanoparticle (e.g., the outer surface of the shell of a SERS nanoparticle having a core-shell structure), wherein at least one of the blocking agent and the specific binding member includes a fluorescent label. Stably associating a blocking agent may include directly binding the blocking agent to the surface of the SERS nanoparticle, or directly binding the blocking agent to a specific binding member that is stably associated with the surface of the SERS nanoparticle. Stably associating a specific binding member may include directly binding the specific binding member to the surface of the SERS nanoparticle, or directly binding the specific binding member to a blocking agent that is stably associated with the surface of the SERS nanoparticle. The methods include associating the fluorescent label to the surface of the SERS nanoparticle using any combination of the fluorescent label, and optionally a blocking agent and/or specific binding member, in any desirable configuration. In certain aspects, both of the blocking agent and the specific binding member include a fluorescent label.

Any suitable synthetic strategies for stably associating the fluorescent label to a surface of the SERS nanoparticle may be employed. For example, the surface of the SERS nanoparticle may be functionalized (or “activated”/“derivatized”) with reactive groups to which the fluorescent label, a blocking agent, a specific binding member, or any combination thereof, may bind to become directly bound to the surface of the SERS nanoparticle. The surface may be functionalized with any useful/convenient reactive group, including but not limited to thiol groups (—SH), amine groups (—NH₂), carboxyl groups (—COO), and/or the like.

Any desirable component of the fluorescently labeled SERS nanoparticle (e.g., the surface thereof, the fluorescent label, a binding agent and/or a specific binding member) may be bound to a second desirable component by reacting a first portion of a crosslinker molecule with a first portion of one of the components, and subsequently reacting a second portion of the component to a second component. Bioconjugation strategies that find use in binding any desirable components of the fluorescently labeled SERS nanoparticles of the present disclosure are described in Hermanson, “Bioconjugate Techniques,” Academic Press, 2nd edition, Apr. 1, 2008, Haugland, 1995, Methods Mol. Biol. 45:205-21; Brinkley, 1992, Bioconjugate Chemistry 3:2, and elsewhere.

According to embodiments in which the SERS nanoparticle has a core-shell structure and the shell is a silica (SiO₂) shell, the shell surface silanol groups may be functionalized. The silica shell can be functionalized to bear free thiol/sulfhydryl groups either during the course of making the SiO₂ shell (“direct modification”), or after the shell has been completely formed (“post-modification”). Reactions for the modification of silanol groups are known and include, but are not limited to, modification of the SiO₂ surface to present amines (e.g., by reaction with aminopropyl trimethoxysilane (APTMS)) or ethoxides (e.g., by reaction with 3-glycidyloxypropyl-trimethoxysilane (GPTMS)). Reagents also exist to incorporate sulfhydryls, carboxyl and other useful reactive groups for conjugation.

The fluorescent label, a blocking agent, or a specific binding member may already include a functional group useful for reacting with a reactive group present on the surface of the SERS nanoparticle, or such a functional group may be provided to the fluorescent label, a blocking agent, and/or a specific binding member. Functional groups that may be used to bind components of the fluorescently labeled SERS nanoparticles include, but are not limited to, active esters, isocyanates, imidoesters, hydrazides, amino groups, aldehydes, ketones, photoreactive groups, maleimide groups, alpha-halo-acetyl groups, epoxides, azirdines, and the like. Reagents such as iodoacetamides, maleimides, benzylic halides and bromomethylketones react by S-alkylation of thiols to generate stable thioether products. For example, at pH 6.5-7.5, maleimide groups react with sulfhydryl groups to form stable thioether bonds. Arylating reagents such as NBD halides react with thiols or amines by a similar substitution of the aromatic halide by the nucleophile. Because the thiolate anion is a better nucleophile than the neutral thiol, cysteine is more reactive above its pKₐ (˜8.3, depending on protein structural context). Thiols also react with certain amine-reactive reagents, including isothiocyanates and succinimidyl esters. The TS-Link series of reagents are available for reversible thiol modification.

With respect to amine reactive groups, primary amines exist at the N-terminus of polypeptide chains and in the side-chain of lysine (Lys, K) amino acid residues. Among the available functional groups in typical biological or protein samples, primary amines are especially nucleophilic, making them ready targets for conjugation with several reactive groups. For example, NHS esters are reactive groups formed by carbodiimide-activation of carboxylate molecules. NHS ester-activated crosslinkers and labeling compounds react with primary amines in physiologic to slightly alkaline conditions (pH 7.2 to 9) to yield stable amide bonds. The reaction releases N-hydroxysuccinimide (NHS). Also by way of example, imidoester crosslinkers react with primary amines to form amidine bonds. Imidoester crosslinkers react rapidly with amines at alkaline pH but have short half-lives. As the pH becomes more alkaline, the half-life and reactivity with amines increases. As such, crosslinking is more efficient when performed at pH 10 than at pH 8. Reaction conditions below pH 10 may result in side reactions, although amidine formation is favored between pH 8-10.

Numerous other synthetic chemical groups will form chemical bonds with primary amines, including but not limited to, isothiocyanates, isocyanates, acyl azides, sulfonyl chlorides, aldehydes, glyoxals, epoxides, oxiranes, carbonates, aryl halides, carbodiimides, anhydrides, and fluorophenyl esters. Such groups conjugate to amines by either acylation or alkylation.

In certain aspects, stably associating the fluorescent label to the surface of the SERS nanoparticle is carried out under aqueous conditions. The present inventors have found that maximum preservation of Raman signal intensity of the Raman reporters of the fluorescently labeled SERS nanoparticles is achieved when the fluorescent label is stably associated with the surface using aqueous chemistry, e.g., to prevent diffusion and/or solubilization of shell-encapsulated Raman reporter molecules from the surface of a metal core of a SERS nanoparticle having a core-shell structure. Functionalization chemistry suitable for aqueous conditions includes, but is not limited to, Sulfo-SMCC (a water soluble analog of SMCC), which may be used to functionalize the surface of the SERS nanoparticle, the fluorescent label, a blocking agent, a specific binding member, etc.

According to one embodiment, the methods include making the fluorescent SERS nanoparticle shown in FIG. 3. As shown in FIG. 3, the synthesis begins by preparing a SERS nanoparticle having accessible thiol groups on the outer surface of a silica shell, e.g., as described above with reference to FIG. 1, in which the inclusion of (3-mercaptopropyl)trimethoxysilane during production of the silica shell provides thiol functional groups on the external surface thereof. The SERS nanoparticle having accessible thiol groups on the external surface of the shell is then treated with the thiol-free reducing agent Tris(2-carboxyethyl)phosphine (TCEP) at pH 7.4, and subsequently with bovine serum albumin that includes a fluorescent label and a maleimide functional group for reacting to a thiol on the external surface of the nanoparticle. The resulting product is a SERS nanoparticle having fluorescent labeled BSA molecules directly bound to the external surface thereof (e.g., the SERS nanoparticle includes a fluorescent labeled BSA coating). In this embodiment, thiol functional groups are subsequently provided to the blocking agent using 2-iminothiolane (2-IT) under aqueous conditions. Next a fluorescent labeled antibody (in this example, a FITC-labeled anti-CD4 antibody) that includes a polyethylene glycol (PEG) linker and maleimide functional group is bound to the blocking agent via reaction between the thiol on the blocking agent and the maleimide bound to the antibody via the PEG linker. The result is a fluorescently labeled SERS nanoparticle in which fluorescent labels are stably associated with the external surface of a SERS nanoparticle via the blocking agent (here, BSA) directly bound to the surface, as well as the specific binding member (here, an antibody) indirectly bound to the surface via the intervening blocking agent.

Also provided by the present disclosure are methods of preparing SERS active imaging reagents. According to certain embodiments, the methods include providing a SERS nanoparticle including a metallic core (e.g., a gold and/or silver core) and a silica shell formed around the metallic core, where the silica shell includes thiol functional groups on the surface thereof. The methods further include stably associating labeled blocking agents with the surface of the silica shell via the thiol functional groups on the surface of the silica shell to form a SERS nanoparticle coated with labeled blocking agents. The methods further include derivatizing the labeled blocking agents with thiol functional groups, and stably associating labeled specific binding members with the labeled blocking agents via the thiol functional groups of the labeled blocking agents to stably associate the labeled specific binding members with the SERS nanoparticle. The methods further include flow cytometrically purifying the SERS nanoparticle coated with labeled blocking agents and stably associated with the labeled specific binding members to prepare a SERS active imaging reagent.

Any of the blocking agents, specific binding members, labels, synthetic strategies, etc. described elsewhere herein may be employed when practicing the methods of preparing SERS active imaging reagents. In certain aspects, the labeled blocking agents and/or the labeled specific binding members are fluorescently labeled or magnetically labeled.

In certain aspects, flow cytometrically purifying the SERS nanoparticle coated with labeled blocking agents and stably associated with the labeled specific binding members includes purifying, by flow cytometry, such a nanoparticle from nanoparticles that are not coated with labeled blocking agents and/or stably associated with labeled specific binding members, thereby preparing the SERS nanoparticle for use as a SERS active imaging reagent. Any suitable flow cytometric-based purification strategy may be employed. Such strategies include, but are not limited to, those described in the Experimental section below.

### Methods

Aspects further include methods of using the above-described fluorescently labeled SERS nanoparticles. Such nanoparticles may be employed in a variety of different applications, e.g., where it is desirable to label an analyte or a particle, such that the analyte or particle can be detected, monitored, analyzed, counted, sorted, collected/enriched, and the like.

Aspects of the present disclosure include methods of evaluating whether an analyte is present in a sample. The methods include contacting the sample with a fluorescently labeled SERS nanoparticle according to any of the embodiments described elsewhere herein, and assessing the sample for a signal from the fluorescently labeled SERS nanoparticle to evaluate whether the analyte is present in the sample.

As summarized above, the methods include contacting a sample with a fluorescently labeled SERS nanoparticle. The term “sample” as used herein means any fluid suspected of containing one or more individual analytes in suspension at any desired concentration. For example, the sample can be suspected of containing 10¹¹ or less, 10¹⁰ or less, 10⁹ or less, 10⁸ or less, 10⁷ or less, 10⁶ or less, 10⁵ or less, 10⁴ or less, 10³ or less, 500 or less, 100 or less, 10 or less, or one analyte per milliliter. The sample can contain a known number of analyte molecules or an unknown number of analytes.

In practicing the methods of the present disclosure, the sample can be a biological sample. A “biological sample” encompasses a variety of sample types obtained from a subject. The definition encompasses biological fluids (e.g., blood (including blood fractions (e.g., serum, plasma)); and other liquid samples of biological origin (e.g., saliva, urine, bile fluid), including liquid samples in which cells of a tissue of a subject are suspended (optionally after being dissociated from one another). “Blood sample” refers to a biological sample, which is obtained from blood of a subject, and includes whole blood and blood fractions (e.g., plasma or serum) suitable for analysis in the present methods. In general, separation of cellular components and non-cellular components in a blood sample (e.g., by centrifugation) without coagulation provides a blood plasma sample, while such separation of coagulated (clotted) blood provides a blood serum sample. Examples of biological samples of blood include peripheral blood or samples derived from peripheral blood. The definition also includes samples that have been manipulated after their procurement, such as by dilution in an appropriate buffer solution, by treatment with reagents, solubilization, or enrichment for certain components, such as one or more cells, polypeptides, and/or nucleic acids to be assayed. For example, a biological sample (e.g., blood) can be enriched for a fraction containing an analyte(s) of interest. Samples can be fractionated by any number of methods including but not limited to ultracentrifugation, fractionation by fast performance liquid chromatography (FPLC), or precipitation.

Accordingly, in some embodiments the sample is obtained from an in vivo source. In certain embodiments the source of the sample is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (e.g., neonate, infant, juvenile, adolescent, adult, geriatric, etc.), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

In practicing the methods of the present disclosure, the sample can be a non-biological sample. For example, a sample may include a suspension of non-biological particles obtained from, e.g., soil, food, water, and the like. Non-biological samples of interest include samples for use in environmental testing, as described more fully herein.

The sample size itself may also vary. In certain embodiments, a sample includes 100 μl or less of fluid, such as 50 μl or less, including about 5 to 50 μl of fluid. A sample may be obtained by, for example, a finger prick (e.g., the sample includes a single fingerstick blood drop). In yet other embodiments, the sample size may be much larger, e.g., 100 μl or greater, such as 500 μl or greater, including 1 ml or greater, e.g., 5 ml or greater, such as where several milliliters of sample are drawn from the circulation of a subject using a suitable blood drawing device (e.g., a syringe). Any convenient means of acquiring a sample may be used in practicing the subject methods.

The conditions under which the sample is contacted with the fluorescently labeled SERS nanoparticle may vary, e.g., according to the type of specific binding agent (if any) that is stably associated to a surface of the SERS nanoparticle. For example, when a specific binding member is present and the specific binding member is a nucleic acid (e.g., an oligonucleotide), the contacting may occur under hybridization conditions such that the nucleic acid specifically hybridizes to the target analyte of interest (e.g., a complementary nucleic acid). By “hybridization conditions” is meant conditions in which primers of the collection of pseudo-random primers hybridize to corresponding nucleic acids in the nucleic acid sample in a sequence-specific manner. Whether a primer specifically hybridizes to a nucleic acid is determined by such factors as the degree and length of complementarity between the specific binding member and the target nucleic acid, and the temperature at which the hybridization occurs, which may be informed by the melting temperature (T_{M}) of the region of the specific binding member that is complementary to the corresponding nucleic acid in the sample. In certain aspects, the melting temperature is a predicted average melting temperature of the collection of primers. The melting temperature refers to the temperature at which half of the primer-nucleic acid duplexes remain hybridized and half of the duplexes dissociate into single strands. The Tm of a duplex may be experimentally determined or predicted using the following formula Tm=81.5+16.6(log₁₀[Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict the Tₘ of primer-nucleic acid duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, Part I, chapter 2, “Overview of principles of hybridization and the strategy of nucleic acid probe assays,” Elsevier (1993).

In certain aspects, the fluorescently labeled SERS nanoparticle includes a specific binding member stably associated with a surface of the SERS nanoparticle (e.g., the outer surface of the shell of a SERS nanoparticle having a core-shell structure), and the specific binding member is an antibody (e.g., an unlabeled antibody, or an antibody that includes a fluorescent label). According to such aspects, the contacting may occur under conditions sufficient for the antibody to specifically bind an analyte of interest. In certain aspects, the analyte is a cell, where the antibody binds to a protein or other feature on the surface of the cell, e.g., a cell surface antigen, such as a cell surface receptor, an extracellular portion of a cell surface associated protein, or the like. Antibodies that specifically bind to cell surface proteins (e.g., a cell surface protein characteristic of a particular cell type, e.g., a particular type of cell of the immune system, a tumor cell (e.g., a circulating tumor cell), a type of stem cell (e.g., a cancer stem cell (CSC)), etc., or subset of cell types) are known in the art and commercially available. By including such antibodies, the fluorescently labeled SERS nanoparticles of the present disclosure may facilitate the detection, analysis, sorting, and/or collection/enrichment of cells of research or clinical interest, e.g., in a flow cytometer. Such cells of interest include cells of the immune system, tumor cells (e.g., circulating tumor cells), rare or low-copy cells within a large mixed-cell population, stem cells (e.g., a cancer stem cell, such as a HER-2⁺/CD44⁺/CD24^{-/low} breast cancer stem cell), hematopoietic cells, or any other type of cell for which it is desirable to assess whether the cell is present in a sample of interest, and if present, optionally quantitate the number of such cells of interest in the sample.

Binding of a fluorescently labeled SERS nanoparticle via an antibody stably associated therewith to a target analyte of interest may be direct or indirect. For example, indirect methods include contacting the analyte (e.g., a cell) with a primary antibody (e.g., a human antibody, a rodent antibody, etc.), and subsequently contacting the primary antibody with the antibody stably associated with a surface of the fluorescently labeled SERS nanoparticle. In this aspect, the antibody stably associated with a surface of the fluorescently labeled SERS nanoparticle serves as a secondary antibody which binds to the primary antibody or a moiety attached thereto.

Protocols for binding antibodies to target antigens are numerous and may vary depending on the affinity of the antibody for the antigen, the organism from which the antibody originated, and other factors known in the art. Such protocols and factors to consider when contacting the sample with the fluorescently labeled SERS nanoparticle can be found, e.g., in Ausubel, F. M. ed. (2005) Current Protocols in Molecular Biology, Wiley & Sons.

As summarized above, the methods of evaluating whether an analyte is present in a sample include assessing the sample for a signal from the fluorescently labeled SERS nanoparticle to evaluate whether the analyte is present in the sample. Assessing the sample for a signal may include: assaying the sample for a fluorescent signal from the fluorescently labeled SERS nanoparticle (e.g., a signal emanating from the fluorescent label stably associated with the surface of a SERS nanoparticle); assaying the sample for a Raman signal from the fluorescently labeled SERS nanoparticle (e.g., a Raman signal emanating from a Raman reporter of the fluorescently labeled SERS nanoparticle); or both.

According to certain embodiments, the assaying is carried out in a flow cytometer. Detecting an analyte of interest (e.g., a cell, such as a circulating tumor cell) in a flow cytometer may include exciting the fluorescent label of the fluorescently labeled SERS nanoparticle with one or more lasers at an interrogation point of the flow cytometer, and subsequently detecting fluorescence emission from the label using one or more optical detectors. It may be desirable, in addition to detecting the analyte, to determine the number of analytes (e.g., cells) labeled with fluorescently labeled SERS nanoparticles of the present disclosure, or utilizing the nanoparticles for the purpose of sorting the analytes. Accordingly, in one embodiment, the methods further include processing the sample (e.g., counting, sorting, or counting and sorting the analytes of interest) by flow cytometry. In one aspect, the analyte to be detected, counted and/or sorted is a cell.

In detecting, counting and/or sorting analytes labeled with the fluorescently labeled SERS nanoparticles of the present disclosure, a liquid medium comprising the analytes is introduced into the flow path of the flow cytometer. When in the flow path, the analytes are passed substantially one at a time through one or more sensing regions (e.g., an interrogation point), where each of the analytes is exposed individually to a source of light at a single wavelength and measurements of light scatter parameters and/or fluorescent emissions as desired (e.g., two or more light scatter parameters and measurements of one or more fluorescent emissions) are separately recorded for each analyte. The data recorded for each analyte is analyzed in real time or stored in a data storage and analysis means, such as a computer, as desired. U.S. Pat. No. 4,284,412 describes the configuration and use of a typical flow cytometer equipped with a single light source, while U.S. Pat. No. 4,727,020 describes the configuration and use of a flow cytometer equipped with two light sources. The disclosures of these patents are herein incorporated by reference in their entireties for all purposes. Flow cytometers having more than two light sources may also be employed.

More specifically, in a flow cytometer, the analytes are passed, in suspension, substantially one at a time in a flow path through one or more sensing regions (or “interrogation points”) where in each region each analyte is illuminated by an energy source. The energy source may include an illuminator that emits light of a single wavelength, such as that provided by a laser (e.g., He/Ne or argon) or a mercury arc lamp with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 535 nm, 635 nm, and the like.

In series with a sensing region, detectors, e.g., light collectors, such as photomultiplier tubes (or “PMT”), are used to record light that passes through each analyte (generally referred to as forward light scatter), light that is reflected orthogonal to the direction of the flow of the analytes through the sensing region (generally referred to as orthogonal or side light scatter) and fluorescent light emitted from the labeled analyte, as the analyte passes through the sensing region and is illuminated by the energy source. Each of forward light scatter (or FSC), orthogonal light scatter (SSC), and fluorescence emissions (FL1, FL2, etc.) comprise a separate parameter for each analyte (or each “event”). Thus, for example, two, three or four parameters can be collected (and recorded) from an analyte labeled with two different fluorescent labels.

Accordingly, in flow cytometrically assaying the analytes, the analytes may be detected and uniquely identified by exposing the particles to excitation light and measuring the fluorescence of each analyte in one or more detection channels, as desired. The excitation light may be from one or more light sources and may be either narrow or broadband. Examples of excitation light sources include lasers, light emitting diodes, and arc lamps. Fluorescence emitted in detection channels used to identify the analytes may be measured following excitation with a single light source, or may be measured separately following excitation with distinct light sources. If separate excitation light sources are used to excite the fluorescent labels, the labels may be selected such that all the labels are excitable by each of the excitation light sources used.

Flow cytometers further include data acquisition, analysis and recording means, such as a computer, wherein multiple data channels record data from each detector for the light scatter and fluorescence emitted by each analyte as it passes through the sensing region. The purpose of the analysis system is to classify and count analytes where each analyte presents itself as a set of digitized parameter values. In flow cytometrically assaying (e.g., detecting, counting and/or sorting) particles in methods of the present disclosure, the flow cytometer may be set to trigger on a selected parameter in order to distinguish the analytes of interest from background and noise. “Trigger” refers to a preset threshold for detection of a parameter. It is typically used as a means for detecting passage of a particle through the laser beam. Detection of an event which exceeds the threshold for the selected parameter triggers acquisition of light scatter and fluorescence data for the analyte. Data is not acquired for analytes or other components in the sample being assayed which cause a response below the threshold. The trigger parameter may be the detection of forward scattered light caused by passage of an analyte through the light beam. The flow cytometer then detects and collects the light scatter and fluorescence data for the analyte.

A particular subpopulation of interest is then further analyzed by “gating” based on the data collected for the entire population. To select an appropriate gate, the data is plotted so as to obtain the best separation of subpopulations possible. This procedure is typically done by plotting forward light scatter (FSC) vs. side (i.e., orthogonal) light scatter (SSC) on a two dimensional dot plot. The flow cytometer operator then selects the desired subpopulation of analytes (i.e., those cells within the gate) and excludes analytes which are not within the gate. Where desired, the operator may select the gate by drawing a line around the desired subpopulation using a cursor on a computer screen. Only those analytes within the gate are then further analyzed by plotting the other parameters for these analytes, such as fluorescence.

Flow cytometric analysis of the analytes, as described above, yields qualitative and quantitative information about the analytes. Where desired, the above analysis yields counts of the analytes of interest in the sample. As such, the above flow cytometric analysis protocol provides data regarding the numbers of one or more different types of analytes in a sample.

Assessing the sample for a signal (e.g., fluorescent and/or Raman signal) from the fluorescently labeled SERS nanoparticle may be carried out using any convenient approach, including non-flow cytometry-based approaches. As just one example, the assessing may be carried out using a sandwich assay. According to one embodiment, a sandwich assay is employed in which the target analyte(s) of interest (e.g., cells of interest, proteins (e.g., antigens) of interest, nucleic acids of interest, etc.) can be immobilized on a solid support indirectly through the binding of the analyte to a specific binding member (e.g., an antibody) that has been immobilized on the solid support. The immobilized target analyte(s) of interest can then be contacted with a fluorescently labeled SERS nanoparticle of the present disclosure that includes a specific binding member, e.g., an antibody that specifically binds the target analyte(s) of interest. In the sandwich assay, the immobilized specific binding member interacts with a separate epitope, nucleic acid sequence, etc. of the analyte of interest than the specific binding member of the fluorescently labeled SERS nanoparticle, resulting in the analyte being sandwiched between the solid support and the nanoparticle. Assessing the reaction area for fluorescent and/or Raman signals may then be carried out to evaluate whether the analyte is present in the sample. Further details regarding nanoparticle-based sandwich assays are found, e.g., in U.S. Patent Application Publication Nos. US2008/0305489, US2011/0172523 and US2012/0164624, the disclosures of which are incorporated herein in their entireties for all purposes.

Aspects of the methods of the present disclosure include the multiplex analysis of two or more distinct particles in a sample and/or the multiplex analysis of single types of particles using two or more distinct types of SERS nanoparticles. For example, “multiplex analysis” may mean that two or more distinct particles are analyzed, e.g., quantitatively. In some instances the number of sets of particles is greater than 2, such as 4 or more, 6 or more, 8 or more, etc., up to 20 or more, e.g., 50 or more. Alternatively, or additionally, “multiplex analysis” may mean that particles of the same type (e.g., a same type of circulating tumor cell, a same type of cancer stem cell, etc.) are analyzed using two or more distinct types of SERS nanoparticles (e.g., fluorescently labeled SERS nanoparticles). In some instances, the number of distinct types of SERS nanoparticles employed to analyze a type of particle is 2, or greater than 2, such as 3 or more, 4 or more, etc., up to 6 or more, e.g., 8 or more.

In certain aspects, multiplexing is facilitated by using a population of fluorescently labeled SERS nanoparticles that includes subpopulations that differ from one another based on the type of Raman reporter present in the different subpopulations. Such subpopulations will emit Raman signals (e.g., fingerprints) unique to the Raman reporter for each subpopulation, and the unique signals may encode information including, but not limited to, the identity of the sample (and accordingly, the subject from which the sample was obtained) that was contacted with the fluorescently labeled SERS nanoparticle prior to the assessing step. In this way, samples from multiple subjects may be simultaneously assessed for the presence of an analyte of interest in an assay format selected by a user of the subject methods, e.g., by flow cytometry, sandwich assay, or any other convenient assay format.

A particle may be analyzed using two or more distinct types of SERS nanoparticles. By way of example, a particular type of cell (e.g., a type of circulating tumor cell, a type of cancer stem cell, etc.) may be analyzed (e.g., quantified) using a population of SERS nanoparticles that includes subpopulations that differ from one another based on a combination of the type of Raman reporter and a type of specific binding member (e.g., an antibody specific for a cell surface antigen of a cell of interest) stably associated with a surface of the nanoparticles. For instance, identifying a cell of interest (e.g., a circulating tumor cell of interest, a cancer stem cell of interest, etc.) may be based on a specific combination of cell surface antigens being present on the surface of the cell. In certain aspects of the present disclosure, a population of SERS nanoparticles may be employed to detect such a cell, where the population includes two or more subpopulations that have a distinct Raman reporter and an antibody specific for one of the cell surface antigens of the specific combination of cell surface antigens. The SERS nanoparticles of such subpopulations may be fluorescently labeled (e.g., the nanoparticles may include a blocking agent that includes a fluorescent label, the antibody may include a fluorescent label, or both). In certain aspects, the SERS nanoparticles of such subpopulations do not include a fluorescent label.

For purposes of illustration, the presence and/or amount of HER-2⁺/CD44⁺/CD24^{-/low} cancer stem cells associated with therapy-resistant breast cancer may be assessed using a nanoparticle population that includes three subpopulations of SERS nanoparticles: (1) a subpopulation that includes a first type of Raman reporter and an antibody that specifically binds HER2; (2) a subpopulation that includes a second type of Raman reporter and an antibody that specifically binds CD44; and (3) a subpopulation that includes a third type of Raman reporter and an antibody that specifically binds CD24. The nanoparticle population may be combined with a sample of interest under specific binding conditions to analyze (e.g., detect, quantify, etc.) the cells of interest using any convenient assay format, an example of which is provided in Example 3 below.

Accordingly, in certain aspects, methods of evaluating whether a cell is present in a sample are provided. The methods include contacting the sample with a population of SERS nanoparticles, wherein the SERS nanoparticles include a core, a Raman reporter adsorbed to the core, and an antibody stably associated with an external surface of the nanoparticles. According to the methods, the population of SERS nanoparticles includes two or more subpopulations of SERS nanoparticles, each subpopulation including a distinct Raman reporter molecule and a distinct antibody that specifically binds an antigen expressed on the surface of the cell. The methods further include assessing the sample for the presence of a cell from which a Raman signal from one or more of the subpopulations emanates, to evaluate whether the cell is present in the sample. According to certain embodiments, the SERS nanoparticles of the population of SERS nanoparticles include a blocking agent stably associated with a surface of the SERS nanoparticles. The blocking agent may be any suitable blocking agent, including any of the blocking agents described hereinabove, e.g., BSA, etc. The SERS nanoparticles of the population of SERS nanoparticles may be fluorescently labeled. For example, the antibody that specifically binds an antigen expressed on the surface of the cell may include a fluorescent label. When the SERS nanoparticles of the population of SERS nanoparticles include a blocking agent, at least one of the blocking agent and the antibody may include a fluorescent label. In certain aspects, both the blocking agent and the antibody include a fluorescent label.

The cell may be any cell for which evaluation is desired. For example, the cell may be a circulating tumor cell, a cancer stem cell, etc. When the cell is a cancer stem cell, in certain aspects the cancer stem cell is a breast cancer stem cell, e.g., a HER-2⁺/CD44⁺/CD24^{-/low} breast cancer stem cell. For example, the methods of evaluating whether a cell is present in a sample may employ: a first subpopulation of SERS nanoparticles, where the SERS nanoparticles of the first subpopulation include a first Raman reporter molecule and an anti-HER-2 antibody stably associated with an external surface of the nanoparticles of the first subpopulation; a second subpopulation of SERS nanoparticles, where the SERS nanoparticles of the second subpopulation include a second Raman reporter molecule and an anti-CD44 antibody stably associated with an external surface of the nanoparticles of the second subpopulation; and a third subpopulation of SERS nanoparticles, where the SERS nanoparticles of the third subpopulation include a first Raman reporter molecule and an anti-CD24 antibody stably associated with an external surface of the nanoparticles of the third subpopulation. The presence or absence of Raman signals emanating from the first, second and third subpopulations indicates the presence or absence of cell surface HER-2, CD44 and CD24 (respectively), and permits evaluation of whether a HER-2⁺/CD44⁺/CD24^{-/low} breast cancer stem cell is present in the sample.

### Utility

The fluorescently labeled SERS nanoparticles and methods of the present disclosure find use in a variety of applications, including but not limited to, applications in which it is desirable to detect an analyte of interest. Applications of interest include, e.g., research applications, clinical applications (e.g., clinical diagnostic applications), etc., and the fluorescently labeled SERS nanoparticles of the present disclosure may be employed in such applications to assess whether an analyte of interest (e.g., a cell, a protein, a nucleic acid, etc.) is present in a sample. In certain aspects, the fluorescently labeled SERS nanoparticles find use in enriching and/or purifying analytes bound to the fluorescently labeled SERS nanoparticles from a large mixed-analyte (e.g., mixed-cell) population by sorting on a flow cytometer. According to certain embodiments, the nanoparticles and methods of the present disclosure find use in further analyzing a particle of interest by SERS imaging, e.g., subsequent to being sorted and/or purified.

In certain aspects, the fluorescently labeled SERS nanoparticles of the present disclosure are highly fluorescent, e.g., due to the inclusion of a plurality of fluorescent blocking agents and/or a plurality of fluorescent specific binding members in the fluorescently labeled SERS nanoparticle. This high level of fluorescence makes such nanoparticles useful as a specific fluorescent labeling agent for detection and enrichment of rare or low-copy circulating cells, such as cancerous or malignant cells, from a large mixed-cell population.

According to certain embodiments, the fluorescently labeled SERS nanoparticles includes blocking agents, e.g., blocking agents coating the external surface of the SERS nanoparticle. The blocking agents result in a significant reduction in the non-specific binding of the nanoparticles compared to counterpart nanoparticles lacking the blocking agents. Such nanoparticles find use in further reducing the number of false positives in detection of rare circulating cells such as cancerous cells (e.g., circulating tumor cells and the like) from a large mixed-cell population.

In addition, the fluorescently labeled SERS nanoparticles and methods of the present disclosure enable staining and sorting of cells of interest without killing the cells, as no fixation or permeabilization is required during the cell staining or sorting processes. As such, the enriched cells obtained from the cell sorting process can be directly used for cell culture to expand the enriched-cell population. The fluorescently labeled SERS nanoparticles also permit analysis of the sorted cells by Raman imaging.

The nanoparticles of the present disclosure circumvent the need for cell enrichment or purification using the magnetic beads, e.g., as a fluorescent labeled antibody included in nanoparticles according to certain aspects provides sufficient fluorescent signal for use in a flow cytometer-cell sorter, thereby obviating the need for the magnetic separation.

In certain aspects, the photostability of the Raman tags permits the use of the fluorescently labeled SERS nanoparticles of the present disclosure in imaging analysis. Unlike the fluorescent dyes, the Raman tags will not photobleach upon repeated and prolonged exposure to a light source (e.g., a laser). As such, the fluorescent label on the surface of the nanoparticles may be used for enrichment of a cell of interest by flow cytometry, and the Raman tag may be used for detection and analysis of the enriched cells by Raman cell imaging and/or Raman microscopy.

In addition, since specific Raman tags have specific and unique spectral signatures, employing a variety of Raman tags permits multiplexing assays. For example, several markers on the surface of a cell of interest can be detected simultaneously using nanoparticles having different Raman tags.

### Kits

As summarize above, the present disclosure provides kits. According to certain embodiments, the kits include a population of fluorescently labeled SERS nanoparticles (e.g., a population of fluorescently labeled SERS nanoparticles according to any of the fluorescently labeled SERS nanoparticles described elsewhere herein), and a container. In certain aspects, the population of fluorescently labeled SERS nanoparticles are present in the container.

According to certain embodiments, the fluorescently labeled SERS nanoparticles of the population include a blocking agent stably associated with a surface of the SERS nanoparticle (e.g., the outer surface of the shell of a SERS nanoparticle having a core-shell structure), where the blocking agent includes a fluorescent label, and where the blocking agent is functionalized with a reaction group, e.g., a reaction group useful for conjugating an additional component (e.g., a specific binding member) to the blocking agent via the reaction group. The blocking agent may include any reaction group of interest, such as a thiol group (—SH), an amine group (—NH₂), a carboxyl group (—COO), or the like. Strategies for functionalizing a blocking reagent with a reaction group are described elsewhere herein and known in the art. See, e.g., Hermanson, “Bioconjugate Techniques,” Academic Press, 2nd edition, Apr. 1, 2008, Haugland, 1995, Methods Mol. Biol. 45:205-21; Brinkley, 1992, Bioconjugate Chemistry 3:2, and elsewhere.

By functionalizing the blocking agent to include a reaction group, a purchaser of the kit may attach a specific binding member (e.g., an antibody, nucleic acid, or the like) to the blocking agent via the reactive group using the any suitable conjugation strategy described elsewhere herein or known in the art. In this way, upon receiving the kit, the purchaser may customize the nanoparticles of the kit to specifically bind to an analyte of particular interest to the purchaser, enabling detection of the analyte of interest in any convenient detection assay format.

Any other components or reagents useful in employing the nanoparticles in an assay of interest may be included in the subject kits. In certain aspects, the kits include a sample processing reagent. Any convenient sample processing reagents may be included. For example, the sample processing reagent may be a blood processing reagent. According to certain embodiments, the sample processing reagent is a diluent.

Components of the subject kits may be present in separate containers, or multiple components may be present in a single container. For example, the population of fluorescently labeled SERS nanoparticles may be provided in a separate container, or in a container that also includes a second component of the kit, e.g., one or more reagents and/or buffers useful for conjugating an additional component (e.g., a specific binding member) to a blocking agent included on the SERS nanoparticles.

The population of fluorescently labeled SERS nanoparticles may be provided in any suitable container. For example, the population may be provided in a single tube (e.g., vial), in one or more wells of a plate (e.g., a 96-well plate, a 384-well plate, etc.), or the like.

In addition to the above-mentioned components, a kit of the present disclosure may further include instructions for using the components of the kit to practice the methods of the present disclosure. The instructions for practicing the subject methods may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1

### Preparation of Fluorescently Labeled SERS Nanoparticles

SERS nanoparticles having a gold core and silica shell as shown in FIG. 1 were used as the starting material to prepare fluorescently labeled SERS nanoparticles. During formation of the thick silica shell around the gold core, APTMS (3-mercaptopropyltrimethoxysilane) was added along with sodium silicate, to introduce thiol functional groups on the surface of the SERS nanoparticles (FIG. 3A). The resulting thiol functional groups were used to anchor fluorescein (FITC)-labeled BSA onto the surface of the nanoparticles (FIG. 3B).

The BSA-coated nanoparticles were further derivatized with a 2-iminothiolane (2-IT) under aqueous conditions to provide thiol functional groups on the BSA coat (FIG. 3C). Finally, an FITC-labeled CD4 antibody is covalently attached to the BSA layer via a PEG linker, resulting in formation of highly fluorescent SERS nanoparticles (FIG. 3D).

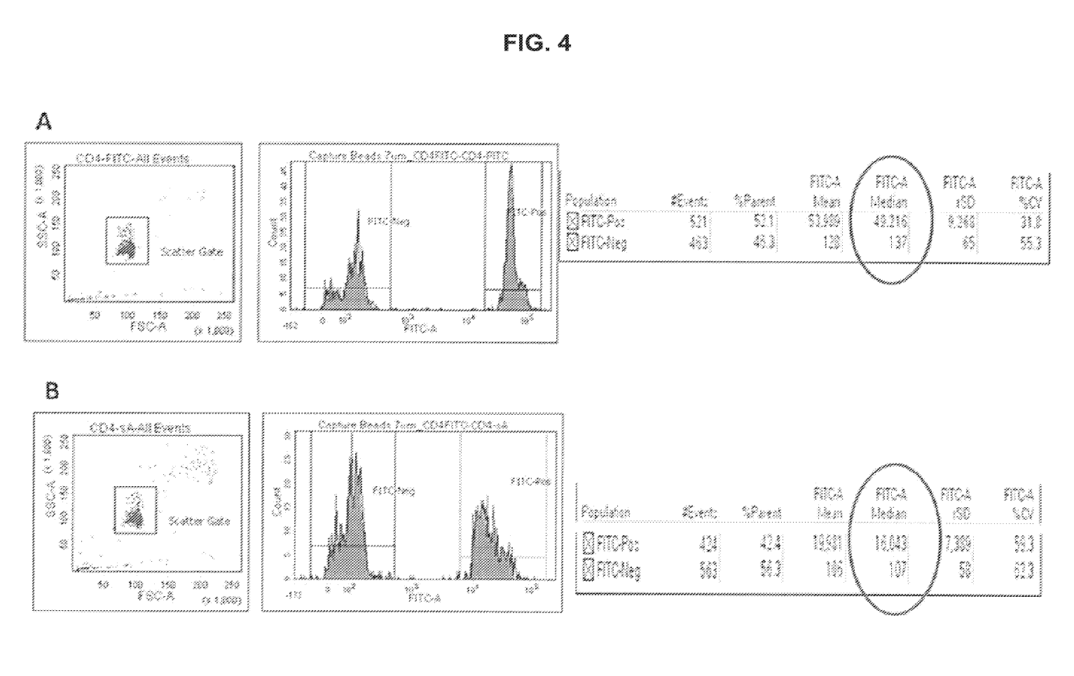

To confirm that the synthesized nanoparticles contain both CD4 antibodies and FITC labels, the particles were captured on 7.5-μm anti-kappa (rat anti-mouse) beads. The anti-kappa beads are coated with rat anti-mouse antibodies that recognize only the mouse kappa-light chain of the CD4 antibody. Therefore, only nanoparticles that contain the conjugated CD4 antibodies on their surface will be captured on these beads. FIG. 4 shows the flow cytometric analysis of such beads after capturing FITC-SERS-CD4 nanoparticles. The fluorescence of the beads (FIG. 4B) in the FITC channel indicates that the nanoparticles contain both CD4 antibodies and FITC labels.

### Example 2

### Reduced Non-Specific Binding of Blocking Reagent-Containing Fluorescently Labeled SERS Nanoparticles

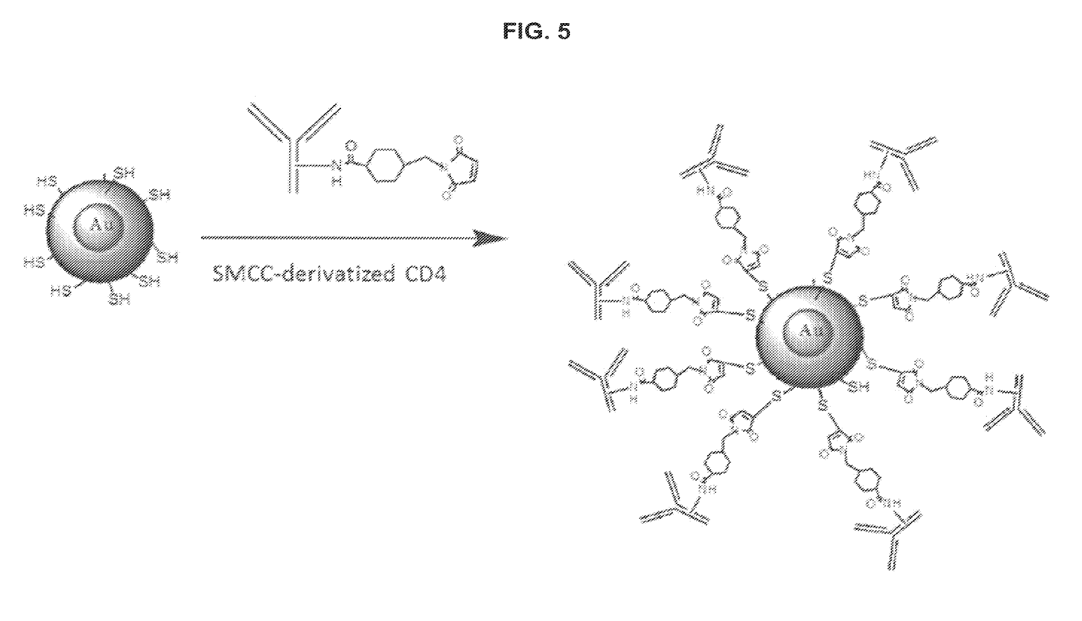

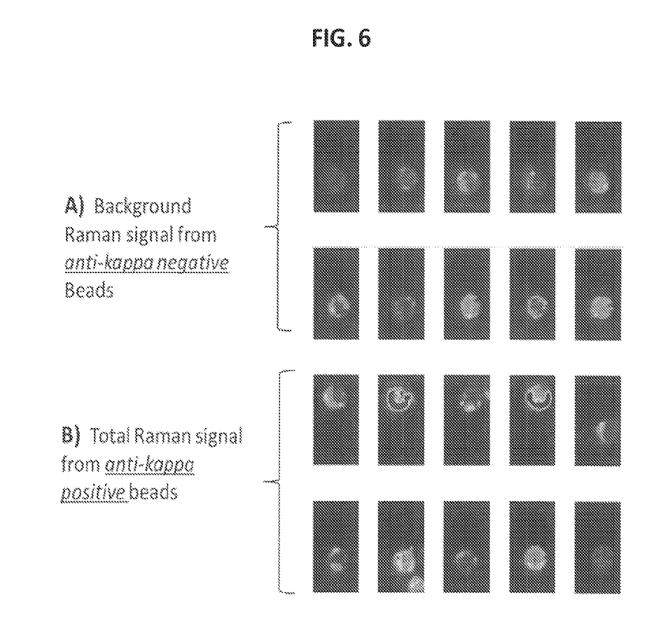

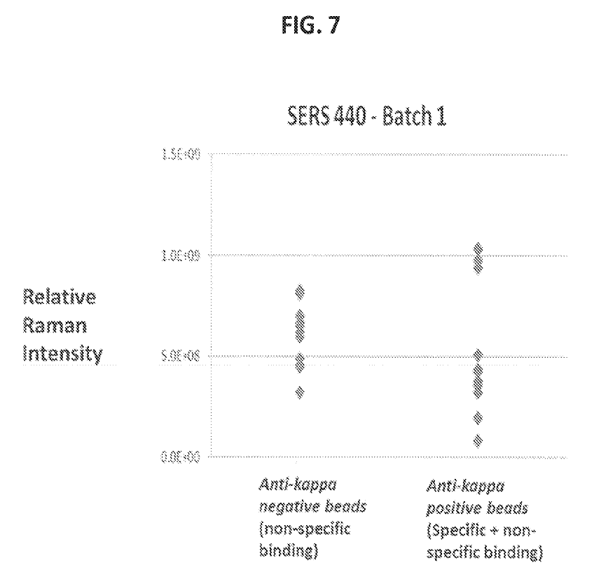

To determine whether the BSA coat of the nanoparticles prepared in Example 1 above reduces non-specific binding of the nanoparticles, a batch of SERS-CD4 nanoparticles with no BSA coating was synthesized for comparison. The chemistry scheme of this synthesis is shown in FIG. 5. As can be seen, the CD4 antibody is directly conjugated to the surface of the SERS nanoparticles with no BSA coating. The resulting nanoparticles were then captured on anti-kappa (negative or positive) beads, and their Raman signals were recorded. FIG. 6 shows the Raman images generated from these beads. The anti-kappa negative beads are polystyrene beads coated with BSA only. Therefore, the Raman signal from these beads represents only the signal from non-specific binding of the nanoparticles. The anti-kappa positive beads are polystyrene beads coated with rat anti-mouse antibodies that recognize only the kappa-light chain of mouse antibody (CD4). As such, the Raman signal from these beads represents the total (specific and non-specific) signals from the captured SERS nanoparticles. The Raman images are shown as the intensity heat map. The red regions indicate the highest intensity of the SERS on a bead, whereas the light and dark blue represent the low and background signals, respectively. FIG. 7 shows a graph representation of the relative intensity of the Raman signals obtained from images in FIG. 5. The graphed data indicates that the background signal from the anti-kappa negative beads (due to non-specific binding of SERS nanoparticles) is high compared to the total signal (specific and non-specific) obtained from the anti-kappa positive beads.

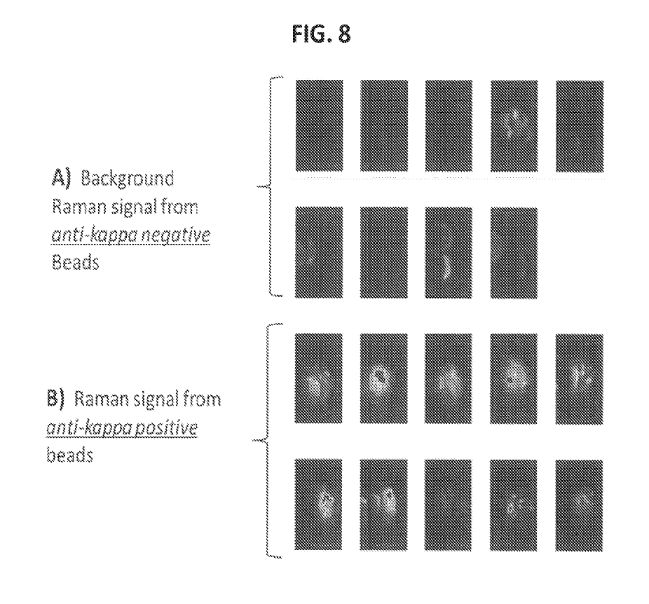

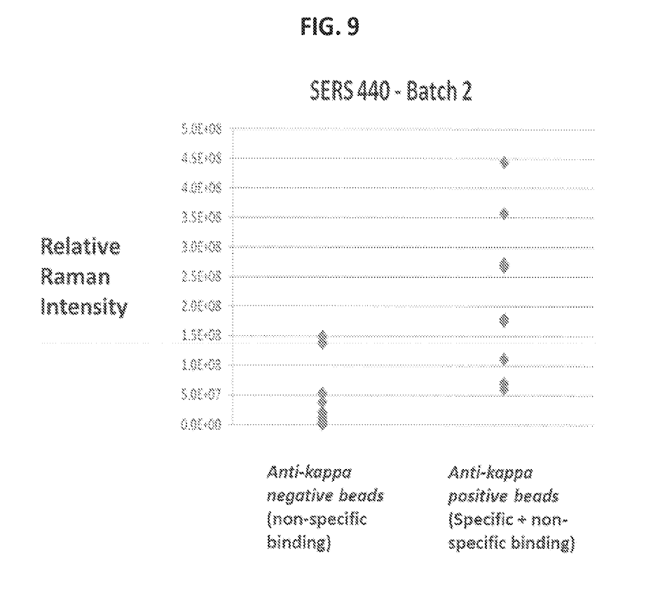

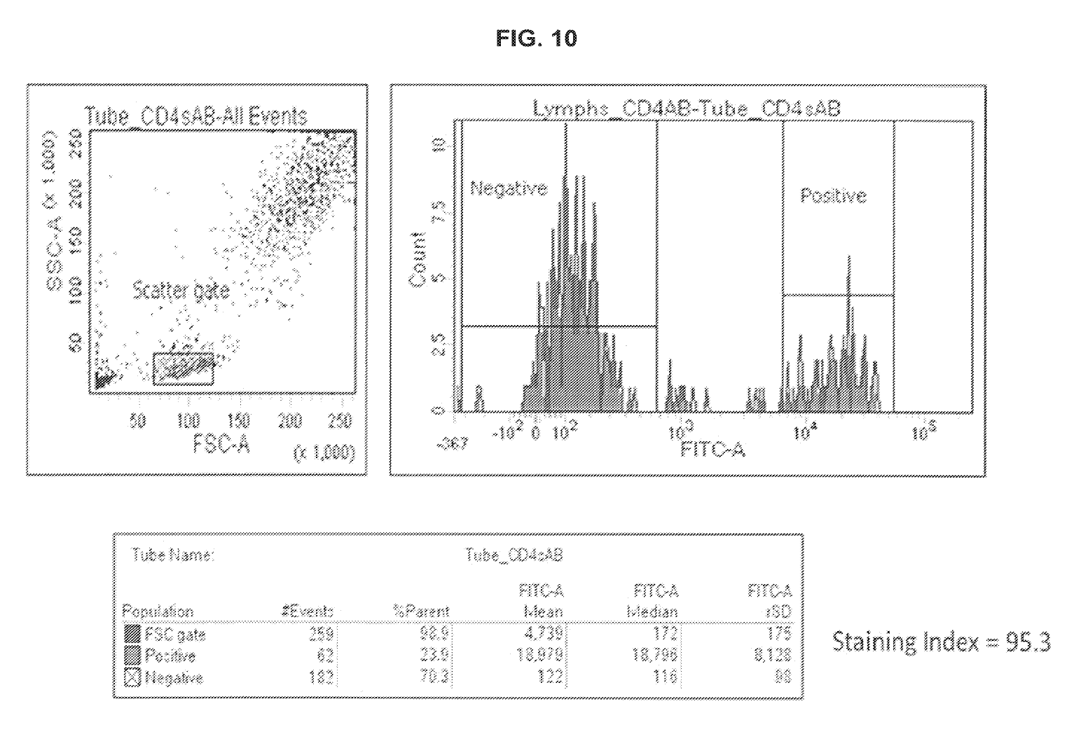

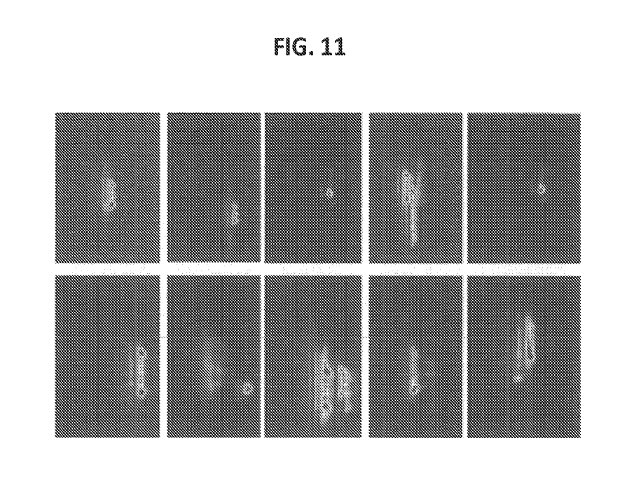

For comparison, the FITC-labeled SERS-CD4 nanoparticles with BSA coating were captured on the anti-kappa (negative or positive) beads, and their Raman signals were recorded, as described above. FIG. 8 shows the Raman images generated from these beads. The relative Raman signal intensities from the images in FIG. 8 are shown as a graph in FIG. 9. The comparison of the graphed data in FIGS. 7 and 9 (as well as visual inspection of the Raman images in FIGS. 6 and 8) indicates that the signal from non-specific binding is considerably lower compared to the total signal (specific and non-specific) when the BSA coating is present on the surface of the SERS nanoparticle.

### Example 3

### Long-Term Imaging of Biomarker Dynamics of Proliferating Cancer Stem Cells

There is increasing evidence that cancer stem cells (CSCs), which possess the ability to self-regenerate, facilitate cancer growth. Chemo- and radiation-therapy resistance of the tumor has been allocated to the same source of the regenerating stem cell pool within the tumor mass. Breast cancer is one of the cancer types where there has been progress in elucidating some of the unique biomarkers to the resistant phenotype, such as HER-2⁻/CD44⁺/CD24^{-/low}. Cellular labels based on surface enhanced Raman scattering (SERS) were employed to further verify the presence and illustrate the development and life-cycle of the CSC's biomarkers. This approach surpasses the capabilities of traditional fluorescent dyes by allowing for multiplexed labeling of individual cells and long-term imaging of any biological sample.

Methods

SERS nanoparticles having a Raman reporter dye adsorbed to a gold core and encased in an SiO2 shell were conjugated with an antibody against HER2, CD44, or CD24. The three resulting types of nanoparticles have a unique Raman signal due to different adsorbed Raman reporter molecules present in the nanoparticle.

HER2-overexpressed breast cancer cell lines were cultured and stained with appropriate SERS nanoparticles for distinct durations of time, after which they were analyzed with a line scan Raman microscope to generate hyperspectral Raman images. The system consisted of a 785 nm excitation laser coupled to a Leica microscope base equipped with a piezo stage and a TE-cooled CCD detector mounted on a grating based spectrometer.

Results

Raman micro-spectral analysis of the HER2-overexpressed cancer cell line allowed for verification and characterization of the performance and stability of SERS nanoparticles. The dynamics of the cellular biomarkers were followed using SERS tags, demonstrating a shorter half-life of HER2 marker as opposed to CD44.

The SERS nanoparticles offer a number of advantages over traditional staining procedures, namely the lack of photobleaching and highly multiplexed detection. Dynamics and lifetimes of the cellular biomarkers may be easily followed using the antibody-modified SERS nanoparticles. Information acquired paves the way for the development of targeted therapies to inhibit cancer metastasis and ultimately enhance patient survival.

Notwithstanding the appended clauses, the disclosure set forth herein is also defined by the following clauses:
1. A fluorescently labeled SERS nanoparticle, the nanoparticle comprising:
   a SERS nanoparticle; and
   a fluorescent label stably associated with a surface of the SERS nanoparticle;
   wherein the SERS nanoparticle comprises a blocking agent stably associated with a surface of the SERS nanoparticle.
2. The fluorescently labeled SERS nanoparticle according to Clause 1, wherein the nanoparticle comprises a specific binding member stably associated with a surface of the SERS nanoparticle.
3. The fluorescently labeled SERS nanoparticle according to Clause 2, wherein at least one of the blocking agent and the specific binding member comprises a fluorescent label.
4. The fluorescently labeled SERS nanoparticle according to Clause 2, wherein both of the blocking agent and the specific binding member comprise a fluorescent label.
5. The fluorescently labeled SERS nanoparticle according to any one of Clauses 1 to 4, wherein the blocking agent is a protein, a nucleic acid, a carbohydrate, a natural molecule or polymer, a synthetic molecule or polymer, or a fluorescent molecule or polymer.
6. The fluorescently labeled SERS nanoparticle according to Clause 5, wherein the protein is a serum protein.
7. The fluorescently labeled SERS nanoparticle according to Clause 6, wherein the serum protein is an albumin.
8. The fluorescently labeled SERS nanoparticle according to Clause 7, wherein the albumin is a bovine serum albumin (BSA).
9. The fluorescently labeled SERS nanoparticle according to Clause 1, wherein the nanoparticle comprises a specific binding member stably associated with a surface of the SERS nanoparticle.
10. The fluorescent labeled SERS nanoparticle according to Clause 9, wherein at least one of the SERS nanoparticle and the specific binding member comprises a fluorescent label bound directly thereto.
11. The fluorescently labeled SERS nanoparticle according to Clause 9, wherein both of the SERS nanoparticle and the specific binding member comprise a fluorescent label bound directly thereto.
12. The fluorescently labeled SERS nanoparticle according to Clause 1, wherein the SERS nanoparticle comprises a fluorescent label bound directly thereto.
13. The fluorescently labeled SERS nanoparticle according to any one of Clauses 2 to 11, wherein the specific binding member comprises an antibody or binding fragment thereof.
14. The fluorescently labeled SERS nanoparticle according to any one of Clauses 1 to 13, wherein the SERS nanoparticle is spherical, spheroid, rod-shaped, or plane-shaped.
15. The fluorescently labeled SERS nanoparticle according to Clause 14, wherein the SERS nanoparticle is spherical or spheroid and has a diameter of from 50 to 500 nm.
16. A method of evaluating whether an analyte is present in a sample, the method comprising:
   contacting the sample with a fluorescently labeled SERS nanoparticle according to any one of Clauses 1 to 15; and
   assessing the sample for a signal from the fluorescently labeled SERS nanoparticle to evaluate whether the analyte is present in the sample.
17. The method according to Clause 16, wherein the assessing comprises assaying the sample for a fluorescent signal from the fluorescently labeled SERS nanoparticle.
18. The method according to Clauses 16 or 17, wherein the assessing comprises assaying the sample for a Raman signal from the fluorescently labeled SERS nanoparticle.
19. The method according to any one of Clauses 16 to 18, wherein the method comprises processing the sample by flow cytometry.
20. The method according to Clause 19, wherein the processing comprises sorting.
21. The method according to any one of Clauses 16 to 20, wherein the analyte is a cell.
22. The method according to Clause 21, wherein the cell is a tumor cell.
23. The method according to Clause 22, wherein the tumor cell is a circulating tumor cell.
24. The method of Clause 21, wherein the cell is a rare or low-copy cell within a large mixed-cell population.
25. A method of making a fluorescently labeled SERS nanoparticle, the method comprising:
   stably associating a fluorescent label with a surface of a SERS nanoparticle.
26. The method according to Clause 25, wherein stably associating a fluorescent label with a surface of a SERS nanoparticle comprises stably associating a blocking agent and a specific binding member with the surface of the SERS nanoparticle, wherein at least one of the blocking agent and the specific binding member comprises a fluorescent label.
27. The method according to Clause 26, wherein stably associating the blocking agent comprises directly binding the blocking agent to the surface of the SERS nanoparticle.
28. The method according to Clause 27, wherein the stably associating the specific binding member comprises directly binding the specific binding member to the blocking agent.
29. The method according to Clause 26, wherein stably associating the specific binding member comprises directly binding the specific binding member to the surface of the SERS nanoparticle.
30. The method according to any one of Clauses 26 to 29, wherein both of the blocking agent and the specific binding member comprise a fluorescent label.
31. The method according to any one of Clauses 26 to 30, wherein the blocking agent is a protein, a nucleic acid, a carbohydrate, a natural molecule or polymer, a synthetic molecule or polymer, or a fluorescent molecule or polymer.
32. A method of preparing a SERS active imaging reagent, the method comprising:
   providing a SERS nanoparticle comprising a metallic core and a silica shell formed around the metallic core, wherein the silica shell comprises thiol functional groups on the surface of the silica shell;
   stably associating labeled blocking agents with the surface of the silica shell via the thiol functional groups on the surface of the silica shell to form a SERS nanoparticle coated with labeled blocking agents;
   derivatizing the labeled blocking agents with thiol functional groups;
   stably associating labeled specific binding members with the labeled blocking agents via the thiol functional groups of the labeled blocking agents to stably associate the labeled specific binding members with the SERS nanoparticle; and
   flow cytometrically purifying the SERS nanoparticle coated with labeled blocking agents and stably associated with the labeled specific binding members to prepare a SERS active imaging reagent.
33. The method according to Clause 32, wherein the metallic core comprises gold, silver, or both.
34. The method according to Clause 32 or 33, wherein the blocking agents are proteins, nucleic acids, carbohydrates, natural molecules or polymers, synthetic molecules or polymers, or fluorescent molecules or polymers.
35. The method according to Clause 34, wherein the blocking agents are serum proteins.
36. The method according to Clause 35, wherein the serum proteins are albumins.
37. The method according to Clause 36, wherein the albumins are bovine serum albumins (BSAs).
38. The method according to any one of Clauses 32 to 37, wherein the blocking agents are fluorescently labeled or magnetically labeled.
39. The method according to any one of Clauses 32 to 38, wherein the specific binding members are fluorescently labeled or magnetically labeled.
40. A kit comprising:
   a population of fluorescently labeled SERS nanoparticles according to any one of Clauses 1 to 15; and
   a container.
41. The kit according to Clause 40, wherein the SERS nanoparticles comprise a blocking agent stably associated with a surface of the SERS nanoparticle, wherein the blocking agent comprises a fluorescent label, and wherein the blocking agent is functionalized with a reaction group.
42. The kit according to Clause 41, comprising a conjugation reagent that facilitates conjugation of a specific binding member to the reaction group of the blocking agent.
43. The kit according to any one of Clauses 40 to 42, comprising a sample processing reagent.
44. The kit according to Clause 43, wherein the sample processing reagent is a blood processing reagent.
45. The kit according to Clause 43 or 44, wherein the sample processing reagent is a diluent.
46. A method of evaluating whether a cell is present in a sample, the method comprising:
   contacting the sample with a population of SERS nanoparticles,
      wherein the SERS nanoparticles comprise a core, a Raman reporter adsorbed to the core, and an antibody stably associated with a surface of the nanoparticles,
      wherein the population of SERS nanoparticles comprises two or more subpopulations of SERS nanoparticles, each subpopulation comprising a distinct Raman reporter molecule and a distinct antibody that specifically binds an antigen expressed on the surface of the cell,
   assessing the sample for the presence of a cell from which a Raman signal from one or more of the subpopulations emanates, to evaluate whether the cell is present in the sample.
47. The method according to Clause 46, wherein the cell is a circulating tumor cell.
48. The method according to Clause 46, wherein the cell is a cancer stem cell.
49. The method according to Clause 48, wherein the cancer stem cell is a breast cancer stem cell.
50. The method according to Clause 49, wherein the breast cancer stem cell is a HER-2⁺/CD44⁺/CD24^{-/low} cancer stem cell.
51. The method according to Clause 50, wherein the population of SERS nanoparticles comprises:
   a first subpopulation of SERS nanoparticles, wherein the SERS nanoparticles of the first subpopulation comprise a first Raman reporter molecule and an anti-HER-2 antibody stably associated with an external surface of the nanoparticles of the first subpopulation;
   a second subpopulation of SERS nanoparticles, wherein the SERS nanoparticles of the second subpopulation comprise a second Raman reporter molecule and an anti-CD44 antibody stably associated with an external surface of the nanoparticles of the second subpopulation; and
   a third subpopulation of SERS nanoparticles, wherein the SERS nanoparticles of the third subpopulation comprise a first Raman reporter molecule and an anti-CD24 antibody stably associated with an external surface of the nanoparticles of the third subpopulation.
52. The method according to any one of Clauses 46 to 51, wherein the SERS nanoparticles of the population of SERS nanoparticles comprise a blocking agent stably associated with a surface of the SERS nanoparticles.
53. The method according to Clause 52, wherein at least one of the blocking agent and the antibody comprises a fluorescent label.
54. The method according to Clause 53, wherein both of the blocking agent and the antibody comprise a fluorescent label.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A fluorescently labeled SERS nanoparticle, the nanoparticle comprising:
a SERS nanoparticle; and
a fluorescent label stably associated with a surface of the SERS nanoparticle;
wherein the SERS nanoparticle comprises a blocking agent stably associated with a surface of the SERS nanoparticle.

2. The fluorescently labeled SERS nanoparticle according to claim 1, wherein the nanoparticle comprises a specific binding member stably associated with a surface of the SERS nanoparticle.

3. The fluorescently labeled SERS nanoparticle according to claim 2, wherein at least one of the blocking agent and the specific binding member comprises a fluorescent label.

4. The fluorescently labeled SERS nanoparticle according to claim 3, wherein both of the blocking agent and the specific binding member comprise a fluorescent label.

5. The fluorescently labeled SERS nanoparticle according to claim 1, wherein the blocking agent is a protein, a nucleic acid, a carbohydrate, a natural molecule or polymer, a synthetic molecule or polymer, or a fluorescent molecule or polymer.

6. The fluorescently labeled SERS nanoparticle according to claim 5, wherein the protein is a serum protein.

7. The fluorescently labeled SERS nanoparticle according to claim 6, wherein the serum protein is an albumin.

8. The fluorescently labeled SERS nanoparticle according to claim 7, wherein the albumin is a bovine serum albumin (BSA).

9. The fluorescently labeled SERS nanoparticle according to claim 1, wherein the nanoparticle comprises a specific binding member stably associated with a surface of the SERS nanoparticle.

10. The fluorescent labeled SERS nanoparticle according to claim 9, wherein at least one of the SERS nanoparticle and the specific binding member comprises a fluorescent label bound directly thereto.

11. The fluorescently labeled SERS nanoparticle according to claim 10, wherein both of the SERS nanoparticle and the specific binding member comprise a fluorescent label bound directly thereto.

12. The fluorescently labeled SERS nanoparticle according to claim 1, wherein the SERS nanoparticle comprises a fluorescent label bound directly thereto.

13. The fluorescently labeled SERS nanoparticle according to claim 2, wherein the specific binding member comprises an antibody or binding fragment thereof.

14. The fluorescently labeled SERS nanoparticle according to claim 1, wherein the SERS nanoparticle is spherical, spheroid, rod-shaped, or plane-shaped.

15. A method of evaluating whether an analyte is present in a sample, the method comprising:
contacting the sample with a fluorescently labeled SERS nanoparticle according to claim 1; and
assessing the sample for a signal from the fluorescently labeled SERS nanoparticle to evaluate whether the analyte is present in the sample.
